(19) United States
(12) Patent Application Publication
(10) Pub. No.: US 2012/0259189 A1
(43) Pub. Date: Oct. 11, 2012

(54) CARDIOVASCULAR INDEX ESTIMATION METHODS

(75) Inventors: Richard Jonathan Cohen, Chestnut Hill, MA (US); Tatsuya Arai, Cambridge, MA (US)

(73) Assignee: Massachusetts Institute of Technology, Cambridge, MA (US)

(21) Appl. No.: 13/425,506

(22) Filed: Mar. 21, 2012

Related U.S. Application Data
(60) Provisional application No. 61/472,366, filed on Apr. 6, 2011.

(51) Int. Cl. A61B 5/02; A61B 8/00; A61B 5/024; A61B 5/026; A61B 5/029; A61B 5/021; A61B 5/1455

(52) U.S. Cl. 600/324; 600/508; 600/485; 600/437; 600/528; 600/504; 600/526; 600/500

(57) ABSTRACT

New algorithms to estimate cardiovascular indices by analysis of the arterial blood pressure (ABP) signal. The invention comprises recording and identification of cardiovascular descriptors (including ABP signal, diastolic pressure, systolic pressure, pulse pressure, and end systole), calculation of cardiovascular system parameters, and calculation of aortic blood flow, stroke volume, cardiac output, total peripheral resistance, and characteristic time constant.

This application claims priority to provisional application Ser. No. 61/472,366 filed on Apr. 6, 2011, the contents of which are incorporated herein by reference in their entirety.

### BACKGROUND OF THE INVENTION

This invention relates to methods for estimating cardiovascular indices from a physiological signal such as an arterial blood pressure signal.

In the modern ambulant and clinical medicine, monitoring of cardiovascular indices such as aortic blood flow (ABF), stroke volume (SV), cardiac output (CO), and total peripheral resistance (TPR) is an indispensable function. The most frequently used CO estimation method is thermodilution that involves injecting cold saline through a central venous catheter into right atrium and measuring the temperature change in the pulmonary artery. However, thermodilution requires a pulmonary artery catheterization, which is associated with cardiovascular risks such as carotid artery puncture (when accessing an intra-jugular vein), cardiac arrhythmia, bleeding, embolism, clotting, and infection (Manecke et al., 2002; Vender and Gilbert, 1997). Moreover, thermodilution cannot continuously measure SV on a beat-to-beat basis, and its accuracy is limited (Botero et al., 2004). Therefore, many studies have been devoted to developing minimally or non-invasive methods to monitor cardiovascular indices over the last 100 years (Erlanger and Hooker, 1904; Herd et al., 1966; Jones et al., 1959; Kouchoukos et al., 1970b; Liljestrand and Zander, 1928; Mukkamala et al., 2006; Parlikar et al., 2007; Sun et al., 2009; Verdouw et al., 1975; Wesseling et al., 1983).

Arterial pulse has been used by researchers to estimate the cardiovascular indices because the arterial pulse is readily accessible in general. In particular, analyses of arterial pressure waveforms (rather than directly measurable simple mean, systolic, and diastolic pressure) have been conducted over the last few decades to extract essential information that can be a better indicator of cardiovascular indices than directly measurable ABP values. Such pulse contour methods (PCMs) have been developed by researchers and limitedly used clinically.

Along the line of PCMs, this invention comprises a novel series of concepts to estimate cardiovascular indices (ABF, SV, CO, TPR) by analysis of the ABP signal on a beat-to-beat basis. The invention also includes identification of cardiovascular descriptors such as end systole, which also improves existing cardiovascular index estimation methods.

### SUMMARY OF THE INVENTION

The method according to the invention for estimating cardiovascular indices includes recording a physiological signal and estimating end systole of one or more cardiac cycles by means other than detecting a dicrotic notch. The physiological signal is then processed to estimate the cardiovascular indices. In a preferred embodiment, the physiological signal is an arterial blood pressure signal, a pulmonary arterial blood pressure signal, an ultrasound signal, or a pulse oximetry signal. In this embodiment, the estimating step involves processing a second signal, such as a heart sound signal, an ultrasound signal, or a blood flow signal. Cardiovascular indices may include instantaneous aortic blood flow, cardiac output, stroke volume, characteristic time constant, and total peripheral resistance. In another embodiment, the estimating step includes estimating an end-systole pressure value and using the estimated end-systole pressure value to estimate the time of end-systole. The end-systole pressure value may be estimated from measures of pressure corresponding to the systolic and diastolic phases of a cardiac cycle.

In yet another embodiment, the processing step involves estimating a characteristic time constant. A specific aortic blood flow waveform shape is assumed. Portions of the arterial blood pressure signal may be scaled in amplitude and/or duration. A filter may also be constructed to reduce estimated instantaneous aortic blood flow during diastole. The method of the invention may further comprise estimation of an arterial compliance. Arterial compliance may be estimated from a pulse transit time or velocity.

### BRIEF DESCRIPTION OF THE DRAWING

FIG. 1 is a schematic illustration showing an auto-regressive with exogenous input model.

FIG. 2a is a graph of autoregressive parameters versus time.

FIG. 2b is a graph of impulse response against time of the ARX model obtained from swine radial ABP data.

FIG. 3 is a graph of systolic interval against a preceding RR interval.

FIG. 4 is a schematic illustration showing end-systole defined by means of partial pulse pressure.

FIG. 5 is a graph showing an example of a binned cross plot of measured and calculated stroke volume.

FIG. 6a is a bar graph showing stroke volume estimation.

FIG. 6b is a bar graph illustrating RNMSE for cardiac output.

FIG. 6c is a bar graph showing estimation of TPR.

FIG. 7a is a bar graph of RNMSE for cardiovascular index estimation using different measures applied to femoral arterial blood pressure.

FIG. 7b is a graph of RNMSE for index estimation applied to femoral arterial blood pressure.

FIG. 7c is a bar graph of RNMSE showing cardiovascular index estimation of TPR applied to femoral arterial blood pressure.

FIG. 8a is a graph of RNMSE applied to radial arterial blood pressure for stroke volume estimation.

FIG. 8b is a bar graph of RNMSE for cardiac output estimation applied to radial arterial blood pressure.

FIG. 8c is a bar graph of RNMSE for TPR estimation applied to radial arterial blood pressure.

FIGS. 9a, b and c are graphs of estimated aortic blood flow versus time using central, femoral and radial arterial blood pressure signals, respectively.

### DESCRIPTION OF THE PREFERRED EMBODIMENT

Novel aortic blood flow, stroke volume, cardiac output and total peripheral resistance estimation algorithms according to the invention will now be described.

Modified Herd's Method

Pulse pressure (PP) is the difference between systolic blood pressure (SBP) and diastolic pressure (DBP) and is often used as an indicator of proportional SV (Erlanger and Hooker, 1904). The algorithm is based on the Windkessel model with an impulse ejection of SV. The drawback of the PP method is the distortion of SBP waveforms; it is known that as ABP waveforms propagate through the tapered and bifurcated arterial tree, the SBP increases and the waveform width becomes narrower. The distortion causes increase in systolic pressure and error in SV estimation. To address the issue, Herd et al. used mean arterial pressure (MAP) instead of SBP because MAP and IMP are known to be robust against distortion (Herd et al., 1966). However, the beat interval MAP includes diastolic interval. A longer diastolic interval would result in smaller SV estimate, regardless of the true duration of systole. To overcome the problem, the present inventors used systolic mean pressure instead of beat interval:

$$\frac{SV}{{C}_{a}} = {\int }_{Systole} P ( t ) d t - DBP \qquad ( 1 )$$

Then CO was calculated from the average of SV over 6 minutes, and TPR from the Ohm's law:

MAP=CO×TPR (2)

CO and TPR estimation in the following methods are conducted in the same manner.

Minimum Variance Method

This method is based on the two-parameter Windkessel model. The proportional aortic blood flow of the Windkessel model is a function of ABP and the characteristic time constant τ;

$$\frac{F ( t )}{{C}_{a}} = \frac{d P ( t )}{d t} + \frac{P ( t )}{τ} \qquad ( 3 )$$

where Cₐ is the arterial compliance. SV is an integral of F in time:

$$\begin{matrix}\frac{SV ( t )}{{C}_{a}} = {\int }_{{t}_{D}}^{t} \frac{F ( t )}{{C}_{a}} d t \\ = {\int }_{{t}_{D}}^{t} ( \frac{d P ( t )}{d t} + β P ( t ) ) d t \\ = P ( t ) - P ( {t}_{D} ) + β {\int }_{{t}_{D}}^{t} P ( t ) d t\end{matrix} \qquad ( 4 )$$

where β=1/τ, and t_{D} is the time of the end systole of the preceding beat. Note that SV is given by integrating systolic ABF; the integral of diastolic ABF (t_{S}<t<T) does not contribute to SV. Therefore, SV(t) should be independent of end integral t if t_{S}<t<T. Such β that minimizes variance of SV(t, t_{S}<t<T) should be used in calculating SV.

$$\frac{\partial V}{\partial β} = 0 \\ ⇒ β = \frac{\begin{matrix}( {\int }_{{t}_{D}}^{{t}_{D} + T} ( P ( t ) - P ( {t}_{D} ) ) d t ) {\int }_{{t}_{D}}^{{t}_{D} + T} I ( t ) d t - T {\int }_{{t}_{D}}^{{t}_{D} + T} \\ ( P ( t ) - P ( {t}_{D} ) ) \cdot I ( t ) d t\end{matrix}}{T {\int }_{{t}_{D}}^{{t}_{D} + T} {I ( t )}^{2} d t - {( {\int }_{{t}_{D}}^{{t}_{D} + T} I ( t ) d t )}^{2}} \qquad ( 5 )$$

In obtaining β, multiple beats (17 beats) were used to minimizing outlier values of β. The value of β was applied to the beat in the middle of the 17-beat moving window to calculate SV, and the moving window was shifted on a beat-to-beat basis. The 17-beat window size was empirically found by the inventors to provide the smallest estimation errors over a wide range of physiological conditions.

Pressure Ratio Method

This method is similar to the Minimum Variance Method. The difference is that SV was calculated at end systole (t=t^{ES}) and end diastole (t=t^{ED}) only. Because the aortic flow is zero during diastole, the two SV values should be equal,

$$\begin{matrix}\frac{SV}{{C}_{a}} = {\int }_{{t}_{i - 1}^{ED}}^{{t}_{i}^{ES}} \frac{F ( t )}{{C}_{a}} d t = {P}_{i}^{ES} - {P}_{i - 1}^{ED} + β \cdot {PSI}_{i} \\ = {\int }_{{t}_{i - 1}^{ED}}^{{t}_{i}^{ED}} \frac{F ( t )}{{C}_{a}} d t = {P}_{i}^{ED} - {P}_{i - 1}^{ED} + β \cdot {PI}_{i}\end{matrix} \\ where {PSI}_{i} ≡ {\int }_{{t}_{i - 1}^{ED}}^{{t}_{i}^{ES}} P ( t ) d t and {PI}_{i} ≡ {\int }_{{t}_{i - 1}^{ED}}^{{t}_{i}^{ED}} P ( t ) d t \qquad ( 6 )$$

Therefore, one can calculate τ (inverse of β) and proportional SV by

$$τ = \frac{1}{β} = \frac{{PI}_{i} - {PSI}_{i}}{{P}_{i}^{ES} - {P}_{i}^{ED}} \qquad ( 7 )$$

and the proportional SV of the beat can be calculated as

$$\begin{matrix}\frac{{SV}_{i}}{{C}_{a}} = {\int }_{{t}_{i - 1}^{ED}}^{{t}_{i}^{ES}} \frac{F ( t )}{{C}_{a}} d t = {P}_{i}^{ES} - {P}_{i - 1}^{ED} + \\ \frac{{P}_{i}^{ES} - {P}_{i}^{ED}}{{PI}_{i} - {PSI}_{i}} \cdot {PSI}_{i} \\ = {\int }_{{t}_{i - 1}^{ED}}^{{t}_{i}^{ED}} \frac{F ( t )}{{C}_{a}} d t = {P}_{i}^{ED} - {P}_{i - 1}^{ED} + \\ \frac{{P}_{i}^{ES} - {P}_{i}^{ED}}{{PI}_{i} - {PSI}_{i}} \cdot {PI}_{i}\end{matrix} \qquad ( 8 )$$

Furthermore, using multiple beats to exclude outliers for practical use in obtain in β=1/τ,

$$\begin{matrix}{\sum }_{i = 1}^{N} \frac{SV}{{C}_{a}} = {\sum }_{i = 1}^{N} ( {P}_{i}^{ES} - {P}_{i - 1}^{ED} ) + β \cdot {PSI}_{i - 1}^{N} \\ = {\sum }_{i = 1}^{N} ( {P}_{i}^{ED} - {P}_{i - 1}^{ED} ) + β \cdot {PI}_{i - 1}^{N} = {P}_{N}^{ED} - {P}_{0}^{ED} + \\ β \cdot {PI}_{i - 1}^{N}\end{matrix} \qquad ( 9 )$$

Where N is the number of the total beats used,

$${PSI}_{i}^{N} ≡ {\int }_{{t}_{i - 1}^{ED}}^{{t}_{i + N}^{ES}} P ( t ) d t = {\sum }_{i = 1}^{N} {PSI}_{i} and$$

$${PI}_{i}^{N} ≡ {\int }_{{t}_{i - 1}^{ED}}^{{t}_{i + N}^{ED}} P ( t ) d t = ( {\sum }_{i = 1}^{N} {T}_{i} ) \overline{P}$$

Therefore, one can calculate τ (inverse of β) by

$$τ = \frac{1}{β} = \frac{( {\sum }_{i = 1}^{N} {T}_{i} ) \overline{P} - {\sum }_{i = 1}^{N} {PSI}_{i}}{{\sum }_{i = 1}^{N} ( {P}_{i}^{ES} - {P}_{i - 1}^{ED} ) - ( {P}_{N}^{ED} - {P}_{0}^{ED} )} \qquad ( 10 )$$

The obtained β is used to calculate SV from (8).

Auto-Regressive with Exogenous Input Model

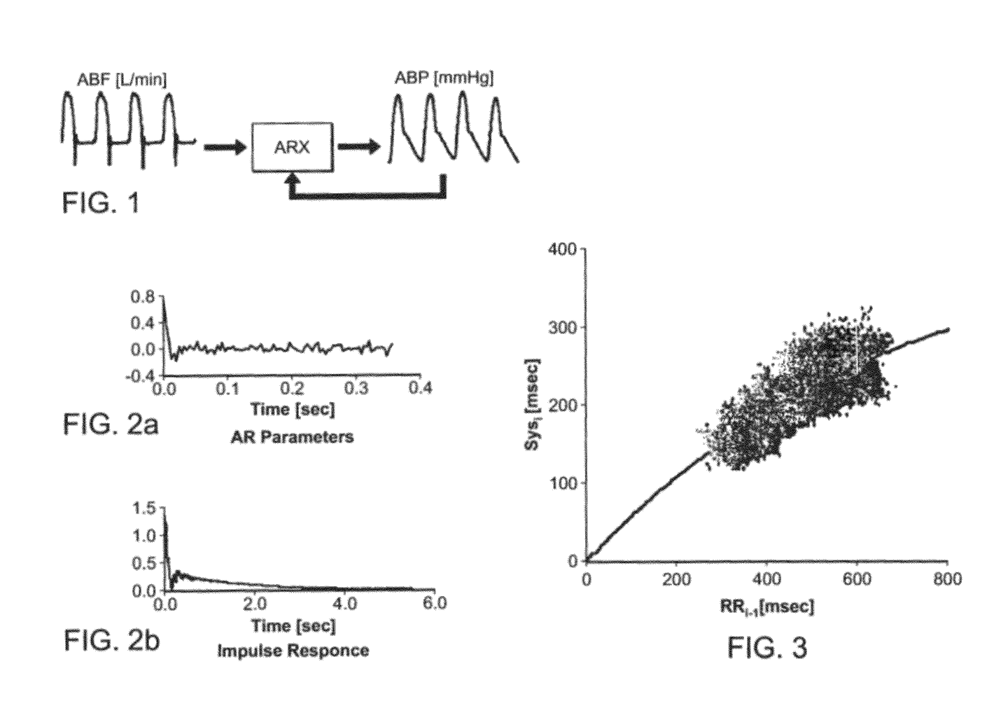

This method takes into account the fact that during diastole the input to the cardiovascular system (i.e., aortic blood flow or ABF) is approximately zero. Over a short time interval, the cardiovascular system can be regarded as a time-invariant system with an input of ABF (F[n]) and output of ABP (P[n]), as illustrated in FIG. 1.

The mathematical model of the system can be described as an Auto-Regressive with Exogenous Input model (ARX model) that relates the ABP values P[n] to the ABF values F[n]:

P[n]=Σⱼ₌₁^{L}a[j]P[n−j]+αF[n] (11)

where the a[j] are the auto-regressive weighting coefficients, L is the coefficient length, and τ is the weighting coefficient for the exogenous input F[n]. Because ABF (system input) is approximately zero during diastole n_{d}:

P[n_{d}]=Σⱼ₌₁^{L}a[j]P[n_{d}−j] (12)

Therefore, the weighting coefficients a[j] are AR coefficients, which can be obtained by solving the matrix equation that involves data from multiple beats:

$$[ \begin{matrix}{P}_{1} [ n ] \\ {P}_{1} [ n + 1 ] \\ ⋮ \\ \underline{{P}_{1} [ n + {N}_{1} - 1 ]} \\ ⋮ \\ \overline{\begin{matrix}{P}_{17} [ n ] \\ {P}_{17} [ n + 1 ] \\ ⋮ \\ {P}_{17} [ n + {N}_{17} - 1 ]\end{matrix}}\end{matrix} ] = [ \begin{matrix}\underline{\begin{matrix}{P}_{1} [ n - 1 ] & … & {P}_{1} [ n + L ] \\ {P}_{1} [ n ] & … & {P}_{1} [ n - L - 1 ] \\ ⋮ & ⋱ & ⋮ \\ {P}_{1} [ n + {N}_{1} - 2 ] & … & {P}_{1} [ n - L + {N}_{1} - 1 ]\end{matrix}} \\ \begin{matrix}⋮ \\ \overline{\begin{matrix}{P}_{17} [ n - 1 ] & … & {P}_{17} [ n - L ] \\ {P}_{17} [ n ] & … & {P}_{17} [ n - L - 1 ] \\ ⋮ & ⋱ & ⋮ \\ {P}_{17} [ n + {N}_{17} - 2 ] & … & {P}_{17} [ n - L + {N}_{17} - 1 ]\end{matrix}}\end{matrix}\end{matrix} ] [ \begin{matrix}a [ 1 ] \\ a [ 2 ] \\ ⋮ \\ a [ L ]\end{matrix} ] \qquad ( 13 )$$

where the Pi[n] are the ABP values in the ith beat and Ni is the number of diastolic samples in the ith beat (1<i<17), A 17 beat moving window size was empirically found to be optimal and was therefore adopted. Note that the elements of the vector item on the left of (4) are diastolic ABP values. The typical AR coefficients a[j] obtained by (4) are shown in FIG. 2a.

The coefficient a can be obtained by taking the average of both sides of (11):

α=(1−Σⱼ₌₁^{L}a[j])MAP/CO (14)

where MAP is mean arterial pressure. MAP/CO can be obtained from Ohm's law (2). TPR can be related to Cₐ and the characteristic time constant of the system r:

τ=Cₐ×TPR (15)

where τ can be obtained by analyzing the exponential decay curve of the impulse response of the system h[n] (FIG. 2b):

h[n]=Σⱼ₌₁^{L}a[j]h[n−j]+δ[n] (16)

Equations (5-7) can be combined to compute α:

α=τ(1Σⱼ₌₁^{L}a[j])/Cₐ (17)

Thus, instantaneous ABF can be expressed as:

$$F [ n ] = \frac{{C}_{a}}{τ ( 1 - {\sum }_{j = 1}^{L} a [ j ] )} ( P [ n ] - {\sum }_{j = 1}^{L} a [ j ] P [ n - j ] ) \qquad ( 18 )$$

The AR coefficient length was chosen to minimize Σa[j]. The integral of F[n] was calculated on a beat-to-beat basis to obtain proportional SV estimates, and the time average of F[n] over six minutes was calculated to obtain a proportional estimate of the CO estimate. Thus, the algorithm presented here provides a comprehensive set of proportional cardiovascular indices (ABF, SV, CO, and TPR) based on an analysis of ABP waveforms.

The aforementioned methods use information of end systole. Researchers have used a dicrotic notch as an indicator of end systole. However, a dicrotic notch is often not available especially in the peripheral ABP signal. Therefore, to advance the SV estimation study, new algorithms to identify end systole without detecting a dicrotic notch were developed.

Exponential Model

The duration of systole (Sys) can be described as a function of the preceding RR interval. Using pilot Yorkshire swine data sets that include measured (true) systolic durations and RR intervals on a beat-to-beat basis, the relation between Sys and RR was found as

Sysᵢ=436(1−exp(−0.0057RRᵢ₋₁^{measured})) (19)

as shown in FIG. 3, and diastolic interval can be calculated as

$$\begin{matrix}{Dia}_{i} = {RR}_{i}^{measured} - {Sys}_{i} \\ = {RR}_{i}^{measured} - 436 ( 1 - exp ( - 0.0057 {R}_{i - 1}^{measured} ) )\end{matrix} \qquad ( 20 )$$

The Exponential Method achieved 6.6% diastolic interval error.

Partial Pulse Pressure Model

An end diastole always comes after a systolic peak. At the end systole, the pressure value is equal to or lower than SBP. When the pressure drops from SBP to a % pulse pressure (PP),

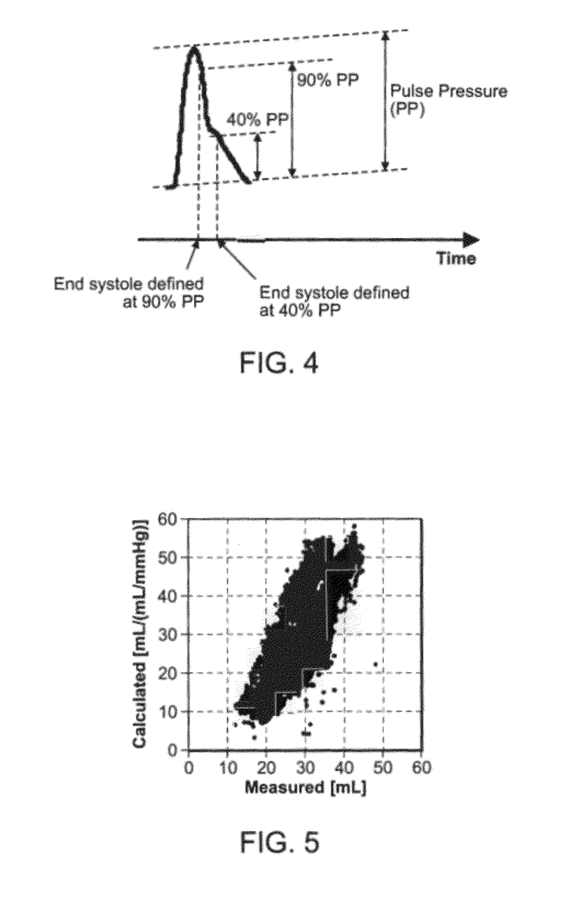

P_{ES}=P_{D}+α(P_{S}−P_{D}) (21)

where P_{ES}, P_{D}, and P_{S} are pressure values at end systole, preceding end diastole, and current beat systole, respectively. As examples, end systoles identified by the 40% PP and 90% PP are shown in FIG. 4. The time stamp of P_{ES} can be regarded as the time of an estimated end systole. The end systole identification errors are summarized in Table 1,

TABLE 1
Summary of diastolic interval errors ± standard deviation (SD).

| | CAP | FAP | RAP |
|---|---|---|---|
| 40% PP | 17.1 ± 11.6 | 5.9 ± 8.0 | 6.0 ± 14.2 |
| 50% PP | 10.1 ± 9.0 | −3.3 ± 5.5 | −1.4 ± 32.5 |
| 60% PP | 1.8 ± 6.9 | −7.4 ± 4.1 | −10.8 ± 6.8 |
| 70% PP | −4.7 ± 4.3 | −10.0 ± 3.9 | −13.8 ± 7.1 |
| 80% PP | −8.2 ± 3.5 | −12.8 ± 3.9 | −17.1 ± 8.2 |
| 90% PP | −11.3 ± 3.8 | −16.3 ± 4.3 | −23.8 ± 11.3 |
| 100% PP | −29.0 ± 21.1 | −31.9 ± 16.9 | −36.4 ± 17.1 |

These end systole identification methods were also applied to the existing SV, CO, and TPR estimation methods Table 2 summarizes the existing cardiovascular index estimation methods that were reported to be competitive in the previous comparison studies (Parlikar, 2007; Sun et al., 2009).

TABLE 2
Existing cardiovascular index estimation methods

| | |
|---|---|
| Pulse Pressure Method (Erlanger and Hooker, 1904) | SV ∝ PP = SBP − DBP |
| Herd's Pulse Pressure Method (Herd et al., 1966) | SV ∝ MAP − DBP |
| Liljestrand-Zander's Method (Liljestrand and Zander, 1928) | $SV = {C}_{a} \times PP ∝ \frac{SBP - DBP}{SBP + DBP}$ |
| Beat-to-Beat Average Model (Parlikar et al., 2007) | $CO ∝ \frac{{P}_{2} - {P}_{1}}{T} + \frac{MAP}{τ} ( Jones et al . , 1959 ; \\ Vender and Gilbert , 1997 )$ |
| Systolic Area (Jones et al., 1959; Verdouw et al., 1975) | $SV ∝ {\int }_{{}_{t} ED}^{{}_{t} ES} P ( t ) d t$ $or$ $SV ∝ {\int }_{{}_{t} ED}^{{}_{t} ES} ( P ( t ) - DBP ) d t$ |
| Corrected Impedance Method (Wesseling et al., 1983) | $SV ∝ ( 163 + HR - 0.48 \cdot MAP ) {\int }_{{}_{t} ED}^{{}_{t} ES} ( P ( t ) - DBP ) \text{?}$ $\text{?} \text{indicates text missing or illegible when filed}$ |
| Kouchoukos Correction (Kouchoukos et al., 1970a) | $SV ∝ ( 1 + \frac{{T}_{S}}{{T}_{D}} ) {\int }_{{}_{t} ED}^{{}_{t} ES} ( P ( t ) - DBP ) d t$ |
| AC Power (Sun et al., 2009) | $SV ∝ \sqrt{\frac{1}{T} {\int }_{T} {( P ( t ) - MAP )}^{2} d t}$ |
| Auto-Regressive Moving Average (ARMA) model (Mukkamaia et al., 2006) | $P [ i ] - {\sum }_{j = 1}^{p} a [ j ] P [ i - j ] + {\sum }_{k = 1}^{q} b [ k ] PP [ i - k ]$ |
| Constant | $SV = {\sum }_{i} {SV}_{i}$ |

Study Protocols

To validate the algorithm described herein and in the provisional application incorporated herein by reference, previously reported (Mukkamala et al., 2006) data from six Yorkshire swine (30-34 kg) recorded under a protocol approved by the MIT Committee on Animal Care were processed and analyzed offline. Table 3 summarizes the physiological ranges of the data sets. Aortic blood flow was recorded using an ultrasonic flow probe (T206 with A-series probes, Transonic Systems, Ithaca, N.Y.) placed around the aortic root. Radial and femoral ABP were measured using a micromanometer-tipped catheter (SPC350, Millar Instruments, Houston, Tex. and an external fluid-filled pressure transducer (TSD1.04A, Biopac Systems, Santa Barbara, Calif.). Aortic blood flow, ECG, and blood pressures were recorded using an A/D conversion system (MP150WSW, Biopac Systems) at a sampling rate of 250 Hz. For more details, please refer to Mukkamala et al. (Mukkamala et al., 2006),

TABLE 3
Summary of cardiovascular parameters of the six swine data sets.

| | Length | CO | SV | Femoral MAP | Radial MAP | HR |
|---|---|---|---|---|---|---|
| ANIMAL | (min) | (L/min) | (mL) | (mmHg) | (mmHg) | (bpm) |
| 1 | 113 | 3.6 +/− 1.0 | 28.4 +/− 5.8 | 63 +/− 19 | 61 +/− 19 | 129 +/− 29 |
| 2 | 97 | 3.2 +/− 0.6 | 25.0 +/− 5.0 | 83 +/− 21 | 73 +/− 20 | 135 +/− 38 |
| 3 | 88 | 4.0 +/− 0.7 | 31.7 +/− 7.1 | 83 +/− 16 | 87 +/− 15 | 133 +/− 32 |
| 4 | 106 | 3.2 +/− 0.6 | 25.2 +/− 4.3 | 89 +/− 19 | 79 +/− 18 | 129 +/− 34 |
| 5 | 90 | 3.3 +/− 0.5 | 26.7 +/− 6.4 | 80 +/− 21 | 85 +/− 19 | 130 +/− 32 |
| 6 | 68 | 3.4 +/− 1.2 | 28.5 +/− 8.1 | 72 +/− 19 | 75 +/− 20 | 130 +/− 26 |
| MEAN | 94 | 3.5 +/− 0.8 | 27.5 +/− 6.7 | 79 +/− 21 | 76 +/− 21 | 131 +/− 32 |

Wide physiological ranges (mean±SD) of cardiac output (CO), stroke volume (SV), femoral and radial mean arterial blood pressure (MAP), and heart rate (HR) were obtained in the six swine data sets.

Error Criterion

Root normalized mean squared error (RNMSE) was adopted as an error criterion.

$$R N M S E = 100 \sqrt{\frac{{\sum }_{n = 1}^{N} {( \frac{Meas - α \cdot Est}{Meas} )}^{2}}{N - {N}_{i}}} \qquad ( 22 )$$

where Meas and Est are measured and estimated values, N is the number of data points, N_{ƒ}is the number of free parameters. Note that all the estimation methods provide SV, CO, and TPR values within a proportionality constant (arterial compliance, α). One of the methods to calculate the proportionality constant α is to divide the mean of the measured values by the mean of the calculated values (i.e., linear regression of the plots shown in FIG. 5. However, this method tends to be driven by majority population of the samples in the middle physiological range (around 30 mL in FIG. 5). The high and low ends that are small in number are weighted less in obtaining the scaling factor α. To overcome this problem, the plots in FIG. 5 were separated into several bins (10 mL interval). The numbers of samples in each bin were considered so that the small number of samples contributes equally. The bin sizes for SV, CO, and TPR estimations are 10 mL, 1 L/min, and 10 mmHg/(L/min), respectively, Thus, the proportionality constants is were selected to minimize the variance of the error:

$${σ}^{2} = \frac{1}{{N}_{k} - 1} {\sum }_{k = 1}^{{N}_{k}} \frac{1}{{N}_{i} ( k )} {\sum }_{i = 1}^{{N}_{i} ( k )} {( \frac{α X ( i , k ) - {SV}_{true} ( i , k )}{{SV}_{true} ( i , k )} )}^{2} \qquad ( 23 )$$

where Nₖ is the number of bins and Ni(k) is number of samples in bin k.

Taking the partial derivative, one can obtain the scaling factor α:

$$\begin{matrix}\frac{\partial }{\partial α} {σ}^{2} = 0 & ( 24 ) \\ ⇒ α = \frac{\frac{1}{{N}_{k} - 1} {\sum }_{k = 1}^{{N}_{k}} \frac{1}{{N}_{i} ( k )} {\sum }_{i = 1}^{{N}_{i} ( k )} \frac{X ( i , k )}{{SV}_{true} ( i , k )}}{\frac{1}{{N}_{k} - 1} {\sum }_{k = 1}^{{N}_{k}} \frac{1}{{N}_{i} ( k )} {\sum }_{i = 1}^{{N}_{i} ( k )} {( \frac{X ( i , k )}{{SV}_{true} ( i , k )} )}^{2}} & ( 25 )\end{matrix}$$

Note that the scaling factor α were obtained for each animal.

For statistical analysis, one-way ANOVA was conducted to find statistical significance among the SV, CO, and TPR estimation methods in conjunction with the end systole identification methods. If a significance difference(s) was found, Scheffé's test was conducted. Statistical significance was defined as p <0.05.

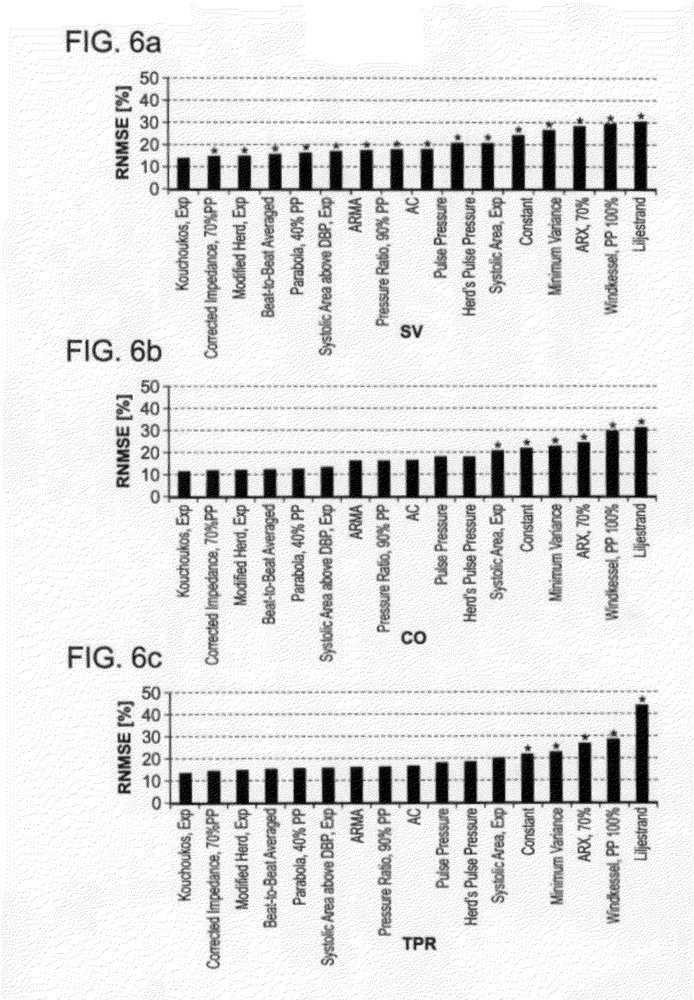

FIG. 6 shows the summary of the SV estimation results by the new and existing methods with their best end systole identification method. Asterisks show the significant difference (p <0.05) from the method with the lowest error. Most of the methods achieved lower errors than the Constant method, indicating that these methods track changes over the wide physiological range.

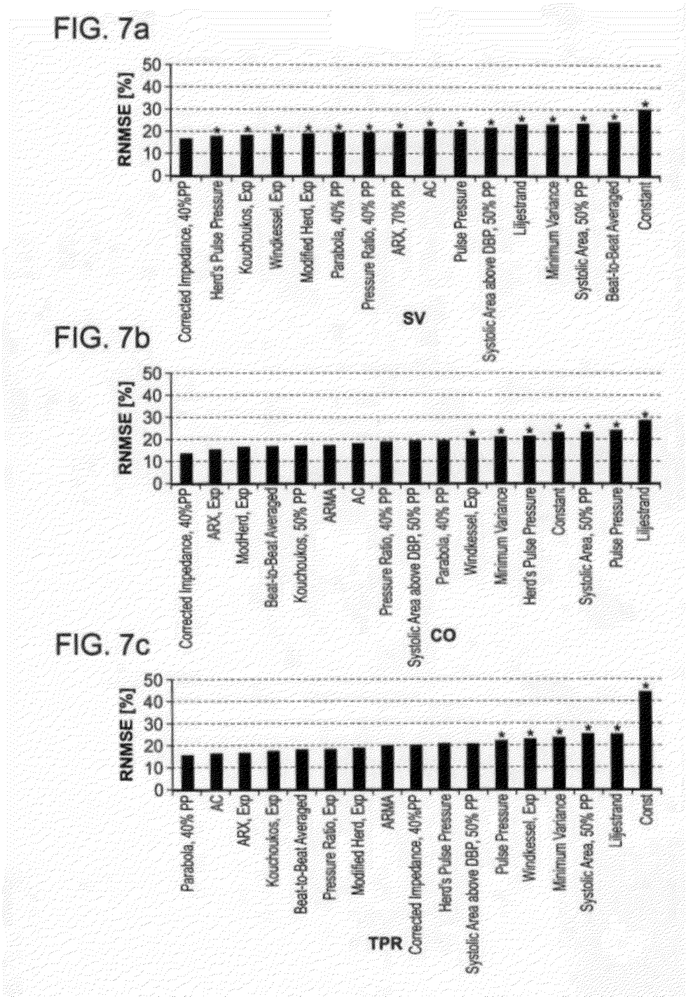

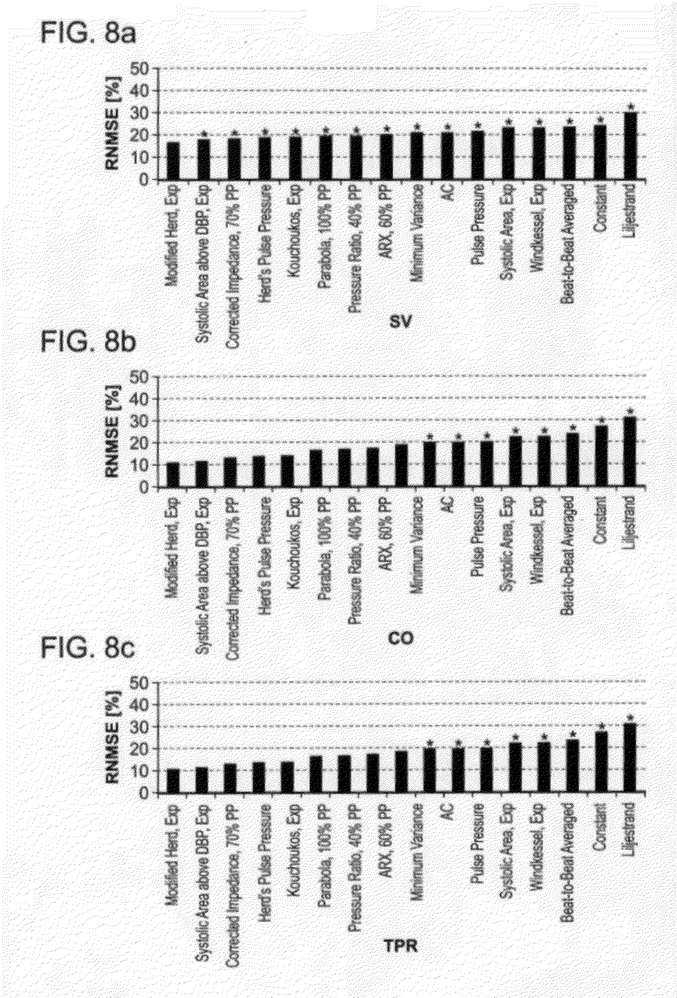

For central ABP, the Corrected Impedance Method with 70% Partial Pulse Pressure as an indicator of end systole resulted in the smallest SV estimation error among the other methods. In a similar manner, FIG. 7 and FIG. 8 show the summary of CO and TPR estimation, respectively. Similar results were seen when using the femoral and radial ABP signal. Among the methods, the Auto-Regressive with Exogenous Input Model, Parabolic Method, Modified Herd's Method, Corrected Impedance Method, and Kouchoukos Correction Method achieved low RNMSEs.

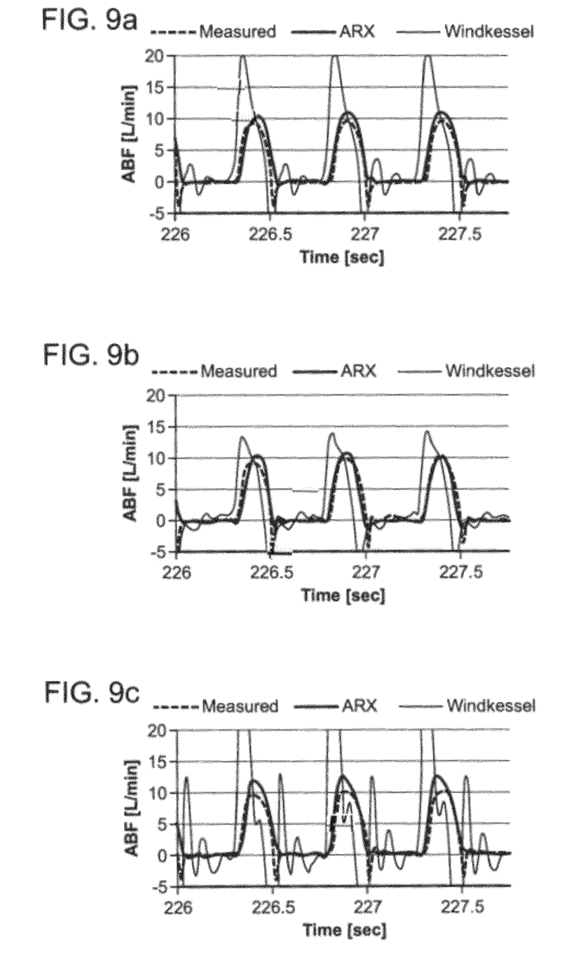

The ABF estimated by the ARX model followed the trend of the measured ABF and presented similar morphology using central (FIG. 9a), femoral (FIG. 9b), and radial ABP signals (FIG. 9c), while the Windkessel ABF presented distorted waveforms.

In this application, new algorithms to estimate cardiovascular indices as well as end systole were introduced. The new methods as well as the existing methods were comprehensively compared. All the methods have their own assumption on the cardiovascular physiology. Development of a cardiovascular index estimation method can be rephrased as developing the most robust method in a variety of physiological ranges and conditions in clinical and research settings.

The parameters of the Wesseling's Corrected Impedance Method were empirically obtained from the human study. The systolic area under the ABP curve above DBP was scaled by a scaling factor that is a function of FIR and MAP. Although they obtained the scaling factor formula from healthy male subjects in their twenties, the method achieved low RNMSEs when applied to the swine data sets, which may indicate that the human and swine cardiovascular systems are similar in terms of applicability of the Windkessel model. Kouchoukos Correction Method includes a simple correction factor (T_{S}/T_{D}) to model run-off blood flow during systole that escapes into the arterial tree without contributing to the ABP signal. Although the correction factor is also empirical, both the Wesseking's and Kouchoukos's methods achieved lower RNMSEs than several theoretical model-based methods. It should be noted that this empirical method could have limitation in accuracy when applied to the monitoring of de-conditioned hearts.

Liljestrand-Zander's method, although it was reported to have the highest agreement with the thermodilution CO in the ICU patient data sets (Sun et al., 2009), did not result in the best method. This could be attributed to the nature of the ICU data sets. Because ABP is maintained during surgeries, one cannot expect as much cardiovascular perturbation in the ABP signals in animal experiments in which vaso-active drugs are used to provide a wide range of cardiovascular indices, Liljestrand-Zander's method divides the PP by the mean of SBP and DBP, which is approximately proportional to MAP. Thus, Liljestrand-Zander's method averages PP by MAP to provide relatively normalized estimates and results in high agreement with the stable ICU thermodilution CO measurements.

The ARX method was also developed and resulted in low errors. The method provides not only proportional SV, CO, and TPR, but also instantaneous ABF waveforms without training data sets or demographic hemodynamic parameters-arguably one of the most comprehensive estimation algorithms to our knowledge. The inventors found that the algorithm achieved 10.8, 12.0, and 10.8% SV RNMSEs if the true end systoles were given. Further development of accurate end systole identification method would lead to more robust ABF, SV, CO, and TPR estimation.

The next step would, be to apply and validate the algorithm with abnormal beats, such as premature beats, and heart failure models. The algorithms should be tested to estimate CO and SV, as well as to reconstruct abnormal ABE and evaluate contractility of the left ventricle. While the empirical methods (Corrected Impedance, for example) would not work for these abnormal heart conditions, sonic of the model-based (cardiovascular physiology-based) methods would work better than empirical methods. Several animal heart failure models have been developed and used for the validation of heart transplantation, left ventricular assist devices, artificial hearts, and so forth (Monnet and Chachques, 2005). Major methods to induce heart failure are (1) pacing-induced—chronic, easy to control (Moe and Armstrong, 1999), (2) ischemia-induced or coronary ligation/occlusion-could be fatal and difficult to control (Einstein and Abdul-Hussein, 1995), and (3) pharmacological (Dixon and Spinale, 2009; Einstein and Abdul-Hussein, 1995; Power and Tonkin, 1999).

Another application of the ARX method and other algorithms includes application to monitoring patients with a severe myocardial infarction. Bi-ventricular pacing that stimulates both left and right ventricles is an option to maintain CO, Currently, central ABP analysis (maximizing dP/dt) and/or short-term echocardiography in the ICU are used to optimize the atrioventricular (A-V) and left and right ventricular pacing delays (Ishikawa et al., 2001; Jansen et al., 2006). However, the optimum delay changes and real-time adaptive cardiac resynchronization are needed. The ARX algorithm can be used to process peripheral ABP waveforms to reconstruct ABE, and such instantaneous ABE waveform information can be used to optimize A-V delay and maximize CO and SV in real time.

In this application, new end systole identification algorithms were also introduced, which advanced not only the new but also the existing SV, CO, and TPR estimation methods. An alternative method for identifying end systoles would be utilization of the S1 and S2 heart sounds that indicate the approximate opening and closing of the aortic valve, respectively. By analyzing the heart sounds and taking the electro-mechanical offsets such as isovolumic contraction period, it would be possible to map systolic/diastolic intervals from the heart sound record to corresponding ABP.

Future work involves application of the methods to human data sets. The challenge is to acquire data sets with a wide physiological range. In the ICU setting, the patients' hemodynamic parameters available to researchers tend to be stable. In such conditions, close-to-constant methods such as Liljestrand would results in low RNMSEs,

This application has introduced new algorithms to estimate cardiovascular indices (SV, CO, and TPR) by analysis of the ABP signal. Algorithms to identify end systole were also introduced and implemented in the existing and new cardiovascular index estimation algorithms. Among the methods. the ARX Model, Parabolic Method, Modified Herd's Method, Corrected Impedance Method, and Kouchoukos Correction Method achieved low RNMSEs. The end systole identification algorithms not only advanced the new but also the existing SV, CO, and TPR estimation algorithms.

The references discussed herein and listed herein are incorporated into this patent application by reference in their entirety. The provisional application from which the present application claims priority is also incorporated herein by reference in its entirety.

### REFERENCES

Botero, M., Kirby, D., Lobato, E. B., Staples, E. D., and Gravenstein, N. (2004). Measurement of cardiac output before and after cardiopulmonary bypass: Comparison among aortic transit-time ultrasound, thermodilution, and noninvasive partial CO2 rebreathing. J Cardiothorac Vase Anesth 18, 563-572.
Dixon, J. A., and Spinale, F. G. (2009). Large animal models of heart failure: a critical link in the translation of basic science to clinical practice. Circ Heart Fail 2, 262-271,
Einstein, R., and Abdul-Hussein, N. (1995). Animal models of heart failure for pharmacological. studies. Clin Exp Pharmacol Physiol 22, 864-868.
Erlanger, J., and Hooker, D. R. (1904). An experimental study of blood-pressure and of pulse-pressure in man (Baltimore, Md., Johns Hopkins Hospital), pp. 1.45-378.
Herd, J. A., Leclair, N. R., and Simon, W. (1966). Arterial pressure pulse contours during hemorrhage in anesthetized dogs. J Appl Physiol 21, 1864-1868.

Ishikawa, T., Sumita, S., Kimura, K., Kikuchi, M., Matsushita, K., Ohkusu, Y., Nakagawa, E, Kosuge, M., Usui, T., and timemura, A. (2001). Optimization of atrioventricular delay and follow-up in a patient with congestive heart failure and with bi-ventricular pacing. Jpn Heart J 42, 781-787.
Jansen, A. H., Bracke, F. A., van Dantzig, J. M., Meijer, A., van der Voort, P. H., Aamoudse, W., van Gelder, B. M., and Peels, K. H. (2006). Correlation of echo-Doppler optimization of atrioventricular delay in cardiac resynchronization therapy with invasive hemodynamics in patients with heart failure secondary to ischemic or idiopathic dilated cardiomyopathy, Am J Cardiol 97, 552-557.
Jones, W. B., Hefner, L. L., Bancroft, W. H., Jr., and Kip, W. (1959). Velocity of blood flow and stroke volume obtained from the pressure pulse. J Clin Invest 38, 2087-2090.
Kouchoukos, N. T., Kerr, A. R., Sheppard, L. C., Ceballos, R., and Kirklin, J. W. (1970a).
Heterograft replacement of the mitral valve: clinical, hemodynamic, and pathological features. Circulation 41, II20-28.

Kouchoukos, N. T., Sheppard, L. C., and McDonald, D. A. (1970b). Estimation of stroke volume in the dog by a pulse contour method. Circ Res 26, 611-623.
Liljestrand, G., and Zander, E. (1928). Vergleichende Bestimmung des Minutenvolumens des Herzens beim Menschen mittels der Stickoxydulmethode und dutch Blutdruckmessung. Zeitschrift fur die gesamte experimentelle Medizin 59, 105-122.
Manecke, G. R, Jr., Brown, J. C., Landau, A. A., Kapelanski, D. P., St Laurent, C. M., and Auger, W. R. (2002). An unusual case of pulmonary artery catheter malfunction. Anesth Analg 95, 302-304, table of contents.
Moe, G. W., and Armstrong, P. (1999). Pacing-induced heart failure: a model to study the mechanism of disease progression and novel therapy in heart failure. Cardiovasc Res 42, 591-599.
Monnet. E., and Chachques, J.C. (2005). Animal models of heart failure: what is new? Ann Thorac Surg 79, 1445-1453.

Mukkamala, R., Reimer, A. T., Hojman, H. M., Mark, R. G., and Cohen, R. J. (2006), Continuous cardiac output monitoring by peripheral blood pressure waveform analysis. IEEE Trans Biomed Eng 53, 459-467.
Parlikar, T. (2007). Modeling and Monitoring of Cardiovascular Dynamics for Patients in Critical Care. In Department of Electrical Engineering and Computer Scienceof Electrical Engineering and Computer Science (Cambridge, Mass. Institute of Technology).
Parlikar, T., Heldt, T., Ranade, G. V., and Verghese, G. (2007). Model-Based Estimation of Cardiac Output and Total Peripheral Resistance. In Computers in Cardiology, pp. 379-381
Power, I. M., and Tonkin, A. M. (1999). Large animal models of heart failure. Aust N Z J Med 29, 395-402,
Sun, J. X., Reisner, A. T., Saeed, M., Heldt, T., and Mark, R. G. (2009). The cardiac output from blood pressure algorithms trial. Crit Care Med 37, 72-80.
Vender, J. S., and Gilbert, H. C. (1997). Monitoring the anesthetized patient, 3 edn (Philadelphia. Lippincott-Raven Publishers).
Verdouw, P. D., Beaune, J., Roelandt, J., and Hugenholtz, P. G. (1975). Stroke volume from central aortic pressure? A critical assessment of the various formulae as to their clinical value. Basic Res Cardiol 70, 377-389,
Wesseling, K. H., De Werr, B., Weber, J. A. P., and Smith, N. T. (1983), A simple device for the continuous measurement of cardiac output. Its model basis and experimental verification, Adv Cardiovasc Phys 5, 16-52.

What is claimed is:

1. Method for estimating cardiovascular indices comprising:
recording a physiological signal;
estimating end-systole of one or more cardiac cycles by means other than detecting a dicrotic notch; and
processing the physiological signal to estimate the cardiovascular indices.

2. The method of claim 1 wherein the physiological signal is an arterial blood pressure signal, a pulmonary arterial blood pressure signal, an ultrasound signal, or a pulse oximetry signal.

3. The method of claim 1 wherein the estimating step involves processing the physiological. signal.

4. The method of claim 1 wherein the estimating step involves processing a signal such as a heart sound signal, an ultrasound signal or a blood flow signal.

5. Method of claim 1 wherein the cardiovascular indices include one or more of the following: instantaneous aortic blood flow, cardiac output, stroke volume, characteristic time constant, and total peripheral resistance.

6. Method of claim 1 wherein the estimating step comprises:
estimating an end-systole pressure value; and
using the estimated end-systole pressure value to estimate the time of end-systole.

7. The method of claim 6 wherein the end-systole pressure value is estimated from measures of pressure corresponding to the systolic and diastolic phases of a cardiac cycle.

8. The method of claim 7 wherein the measures are the peak systolic and end-diastolic pressures.

9. The method of claim 1 where in the processing step involves estimating a characteristic time constant.

10. The method of claim 1 wherein a specific aortic blood flow waveform shape is assumed.

11. The method of claim 1 wherein portions of the arterial blood pressure signal are scaled in amplitude and/or duration.

12. The method of claim 1 further comprising the construction of a filter which reduces estimated instantaneous aortic blood flow during diastole.

13. The method of claim 12 wherein the filter is constructed from an ARX model.

14. The method of claim 1 wherein the arterial pressure signal is obtained from an intra arterial measurement location.

15. The method of claim 1 wherein the arterial pressure signal is obtained from a noninvasive blood pressure measurement device.

16. The method of claim 1 further comprising the estimation of an arterial compliance.

17. The method of claim 16 wherein the arterial compliance is estimated from a pulse transit time or velocity.

18. The method of claim 9 wherein multiple cardiac cycles are processed to estimate the characteristic time constant.

19. The method of claim 1 wherein end-systole of a cardiac cycle is estimated by analyzing the timing of the preceding cardiac cycles.

20. The method of claim 1 wherein the processing step employs the principle that instantaneous aortic blood flow is approximately zero during diastole.

21. Method for estimating cardiovascular indices comprising:
recording an arterial blood pressure signal
processing the arterial blood pressure signal to estimate the instantaneous aortic blood flow.

22. The method of claim 21 furthering comprising estimating from the instantaneous aortic blood flow signal one or more cardiovascular indices.

23. Method of claim 22 wherein the cardiovascular indices include one or more of the following: instantaneous aortic blood flow, cardiac output, stroke volume, characteristic time constant, and total peripheral resistance.
